(19) United States
(12) Patent Application Publication
(10) Pub. No.: US 2014/0294988 A1
(43) Pub. Date: Oct. 2, 2014

(54) SKIN TREATMENT FORMULA FOR PSORIASIS

(71) Applicant: Gaitree Cruz, Hallandale Beach, FL (US)

(72) Inventor: Gaitree Cruz, Hallandale Beach, FL (US)

(21) Appl. No.: 13/850,818

(22) Filed: Mar. 26, 2013

(51) Int. Cl. A61K 36/889; A61K 36/738; A61K 36/185; A61K 31/60; A61K 31/355; A61K 36/58; A61K 36/63; A61K 35/64

(52) U.S. Cl. CPC A61K 36/889; A61K 36/63; A61K 36/738; A61K 35/644; A61K 31/60; A61K 31/355; A61K 36/58; A61K 36/185
USPC 424/539

(57) ABSTRACT

A topical treatment is disclosed for combating psoriasis comprising a combination of coconut oil, cocoa butter, olive oil, beeswax and rose oil. Versions of the formula also may contain neem oil, jojoba oil and/or salicylic acid. The formula is topically applied to the skin after a washing and can be used as preventative measure as well as treatment for an acute flare up.

### BACKGROUND OF THE INVENTION

1. Field of the Invention The present invention relates to dermatological medical therapy, and more particularly, to a topical treatment formula and therapy for psoriasis and related skin conditions.

2. Description of the Related Art

Many medical treatments for a wide range of skin conditions have been designed in the past. None of them, however, includes the unique combination of constituents that symbiotically interact to effectively combat psoriasis and other related skin and dermatological conditions resulting in healthier skin.

Some patents and medical publications describe various attempts at treating psoriasis the closest subject matter provide for a number of more or less complicated features that fail to solve the problem in an efficient and economical way. None of these patents suggest the novel features of the present invention.

3. Background

Psoriasis and related skin conditions are unpredictable and irritating. It is one of the most misunderstood and persistent skin disorder. It is typically characterized by skin cells that multiply many times faster than would be found in normal, healthy skin. As underlying cells reach the skin's surface and die, the large number of cells causes raised red plaques that can be covered with a white scale.

Psoriasis typically occurs on the knees, elbows, and scalp. In some people it can also affect other parts of the body including the torso, palms, and soles of the feet. The symptoms of psoriasis can vary somewhat from person to person.

Some common symptoms for plaque psoriasis, the most common variety of the condition, include: plaques of red skin, often covered with loose, silver-colored scales. These lesions may be itchy and painful. They sometimes crack and bleed. In severe cases, the plaques of irritated skin will grow and merge into one another, covering large areas of the body which complicate the situation.

Disorders of the fingernails and toenails can sometime also occur as a symptom. These may include, for example, discoloration and pitting of the nails. In more severe cases the nails may also begin to crumble or detach from the nail bed. This can cause a painful reaction and puts the patient at increased risk of secondary infections.

Plaques, scales or crust on the scalp may also sometimes occur. Psoriasis can be associated with psoriatic arthritis, which leads to pain and swelling in the joints. The National Psoriasis Foundation estimates that between ten and thirty percent of people with psoriasis also have psoriatic arthritis.

Other forms of psoriasis include pustular psoriasis, characterized by red and scaly skin on the palms of the hands and/or feet with tiny pustules.

Guttate psoriasis, which often starts in childhood or young adulthood, is characterized by small, red spots, mainly on the torso and limbs. Triggers for this type may be respiratory infections, strep throat, tonsillitis, stress, injury to the skin, and use of anti-malarial and beta-blocker medications.

Inverse psoriasis, characterized by bright-red, shiny lesions that appear in skin folds, such as the armpits, groin area, and under the breasts can also occur.

Erythrodermic psoriasis, characterized by periodic, fiery redness of the skin and shedding of scales in sheets. This form of psoriasis, triggered by withdrawal from a systemic psoriasis treatment, severe sunburn, infection, and certain medications, requires immediate medical treatment because it can lead to severe illness.

Psoriasis generally is an uncomfortable and at times disfiguring skin disease that can be difficult and frustrating to treat. The condition frequently comes and goes in periodic cycles of remissions and flare-ups that can continue over a lifetime. While there are medications and other therapies that can help to clear up the patches of red, scaly, thickened skin that are the hallmark of psoriasis, there is no cure.

A multiplicity of external and internal factors ranging from emotional stress and trauma to a streptococcal infection can cause a bout of psoriasis. Recent research has shown that in some cases an abnormality in the body's immune system is one of the key causes of psoriasis. As many as eighty percent of people reported having flare-ups subsequent to an exceptional emotional event, such as the loss of a job or other taxing event. Many physicians believe such external stressors serve as initiators for a genetic defect in immune function.

Damaged skin and certain pharmaceuticals can stress the immune system and aggravate psoriasis. For example, certain types of blood pressure medications (like beta blockers) and hydroxychloroquine (a malaria treatment) tend to show a causal connection between use and flare-ups.

Psoriasis has a significant genetic component and tends to run in families. It may be skip generations; a grandmother and his granddaughter might be affected however the child's mother does not exhibit the condition. Although psoriasis may be stressful and embarrassing, most outbreaks are generally harmless. With careful treatment most of the symptoms generally be partially or totally resolved in several weeks to months.

### SUMMARY OF THE INVENTION

It is one of the main objects of the present invention to provide a topical formula that can clear a patient's skin of existing psoriasis, leaving a soft and smooth surface.

It is another object of this invention to provide a formula that nurtures and heals the skin using only natural ingredients without harsh chemicals.

It is still another object of the present invention to provide a unique formula with natural oils that removes psoriasis plaques, flakes, redness, and relieves itching.

It is yet another object of this invention to provide such a product that is inexpensive to manufacture and maintain while retaining its effectiveness.

Further objects of the invention will be brought out in the following part of the specification, wherein detailed description is for the purpose of fully disclosing the invention without placing limitations thereon.

### BRIEF DESCRIPTION OF THE DRAWINGS

With the above and other related objects in view, the invention consists in the details of construction and combination of parts as will be more fully understood from the following description, when read in conjunction with the accompanying drawings in which:

FIG. 1 is a table showing range and preferred percentage by weight of a version of the formula for severe or basic treatment.

FIG. 2 is a table showing range and preferred percentage by weight of a version of the formula for a maintenance treatment.

FIG. 3 is a table showing range and preferred percentage by weight of a version of the formula for extra moisturizing treatment.

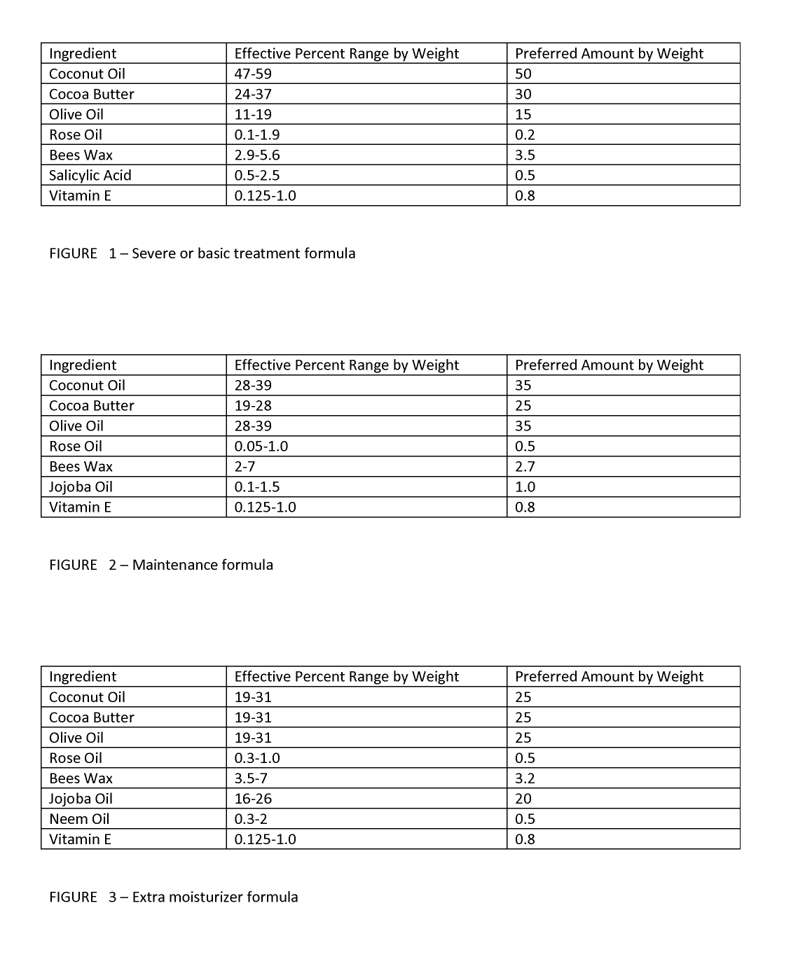

### DETAILED DESCRIPTION OF THE PREFERRED EMBODIMENT

The present invention is a unique formula with natural oils that removes psoriasis plaques, flakes, redness, and itching. It is not a hand lotion but instead it is specifically formulated for removing plaques and is generally most effective only for that purpose.

Generally, the mixture is made available in a two ounce jar and a one ounce roll-on bottle. The one ounce roll-on bottle offers the convenience of pocket or travel size that can be used anywhere discreetly to the areas of psoriasis whenever and wherever it may happen to surface. This also provides easy access for maintaining consistency in application to prevent reoccurrence.

The formula is generally used by first preparing the psoriasis affected skin areas by cleansing with anti-bacterial soap and then completely flushing the area with water. Pat the skin dry with a clean and dry cloth or paper towel to remove most of the water. It is best to avoid rubbing dry. Likewise, use of scents and dyes in drying towels should preferably be avoided. The formula should be applied only to the affected area and massaged in thoroughly with the hands until formula is totally absorbed.

If area affected is frequently washed, such as the hands, it will be necessary to apply after each wash to prevent plaques from reforming and keeping psoriasis manageable. Vigilance in preventing an outbreak from developing is more effective that stopping an outbreak that has already begun to take off.

Diet plays a vital role in managing psoriasis. This formula can be an important part of the course of treatment but will be more effective when multiple avenues of repressing an outbreak are simultaneously carefully followed.

Like many diseases, diet is important. It helps to eat a balanced diet. Limit fried foods and sugary drinks. Eat homegrown fruits and vegetables if possible. In any event, ensure that fruits and vegetables are thoroughly washed before eating to help remove any pesticides that may be on the surface.

The formula works by nourishing the skin to help provide all of the macro and micro nutrients that the skin needs to continue on a natural and healthy life course. The oils, lipids, moisturizers and waxes nourish the skin and reduce inflammation that can lead to irritated skin and ultimately to another outbreak.

Wheeling exercises also are an important part of healing. Wheeling is generally a mechanically aided massage that soothes the muscles, skin and joints. A wheeling device is similar to a rolling pin or a frame roller for painting in that a cylindrical roller affixed to a frame is rotatable about an axis.

The wheel or roller is used to roll over affected areas after they have been cleaned and the treatment formula has been applied. The patient can often roll over themselves but may also need assistance reaching some areas effectively. The wheel is systematically rolled in a patter over the skin to soothe an area during an acute period of inflammation and psoriasis flare ups.

The practitioner treating the patent typically does an initial patient evaluation that includes a full skin assessment, lab work, medical history and plan of care. The plan of care typically includes regimented use of the present formula in any of it several variations, herbal treatments and wheeling exercises.

Continued nursing care includes education and teaching about the underlying skin conditions, alternative medical treatments, holistic treatments and dietary science. The more a patient learns about the condition the better suited they are to participate in monitoring and treating the condition. Patients are also monitored closely by their caregiver to identify and ensure optimal progress and results with the treating physician.

Some patients find the addition of vitamin D applications beneficial in combination with the other elements of this course of treatment. This may maximize the speed and completeness of healing and skin improvement.

It should be noted that in the description of the formulas as presented herein that all percentages are percentages by weight of the total weight of the formula unless otherwise specifically noted. A specific ideal percentage of each ingredient is provided as merely a guide for a highly effective amount. Obviously, the range of weight is provided for each ingredient to show an effective range. A percentage weight outside the specified range may generally effective but to a lesser degree than a weight within the specified range.

When a percent amount is specified it is intended to mean within a decimal place of precision. For example, if 1.9 is specified this is intended to include from about 1.85 through 1.94. Similarly, if 30 is specified then the range is intended to encompass 29.5 through 30.4.

The ingredients may also include purified water in any of the several formulations to total one hundred percent as long as the other constituent ingredients are within the specified range.

In a method of manufacture, the ingredients are made by combining all ingredients and heating to about 140 degrees Fahrenheit to about 160 degrees Fahrenheit. The mixture is then blended thoroughly and then containerized.

A cold blending is also possible. Cold is generally defined as approximately room temperature but may be between about sixty and one-hundred degrees Fahrenheit. The mixture is then thoroughly blended and then containerized.

Now referring to the drawings where figure one shows a table describing the ingredients for a version of a formula for severe or basic treatment. In this mixture coconut oil is the primary ingredient with anywhere between forty-seven and fifty-nine percent with about fifty percent being ideal. Pure cocoa butter is between twenty-four and thirty-seven percent with about thirty percent being ideal. Extra virgin olive oil is between eleven and nineteen percent with about fifteen percent being ideal. Rose oil is between 0.1 and 1.9 percent with about 0.2 percent being ideal. Beeswax is between 2.9 and 5.6 percent with about 3.5 percent being ideal. Salicylic acid is between 0.5 and 2.5 percent with about 0.5 percent being ideal. Vitamin E is optionally included between about 0.125 and 1.0 percent with about 0.8 being ideal.

It should be appreciated that each of the several versions of the formulas identify and list the active ingredients and that other non-active ingredients may also be present. For example, water may be included as an ingredient or other binders, fillers and non-active ingredients. Vitamin E is included in each of the formulas to prevent rancidity and for moisturizing purposes. However, in some patients using the formulas vitamin is not included for particular sensitivities.

Table two shows a version of the formula that is ideal for long term maintenance of the skin to keep psoriasis inflammation from returning. In this mixture coconut oil is the primary ingredient with from between twenty-eight and thirty-nine percent with about thirty-five percent being ideal. Cocoa butter is between nineteen and twenty-eight percent with about twenty-five percent being ideal. Olive oil is between twenty-eight and thirty-nine percent with about thirty-five percent being ideal. Beeswax is between two and seven percent with about 2.7 percent being ideal. Rose oil is between about 0.05 and 1.0 percent with about 0.5 percent being ideal. Jojoba oil is between about 0.1 and 1.5 percent with about one percent being ideal. Vitamin E is optionally included between about 0.125 and 1.0 percent with about 0.8 being ideal.

Table three shows a formulation that is preferred by some users because it provides an extra degree of moisturizing that is beneficial for some patients in some situations. For example, a higher moisturizer may be better suited to dryer or colder environmental conditions or those with inherently drier skin.

In this combination coconut oil, cocoa butter and olive oil are primary ingredients. Coconut oil is between nineteen and thirty-one percent with about twenty-five percent being ideal. Cocoa butter is between nineteen and thirty-one percent with about twenty five percent being ideal. Olive oil is between nineteen and thirty-one percent with about twenty-five percent being ideal. Jojoba oil is between sixteen and twenty-six percent with about twenty percent being ideal. Rose oil is between 0.3 and 1.0 percent with about 0.5 percent being ideal. Neem oil is between 0.3 and two percent with about 0.5 percent being ideal. Beeswax is present between 3.5 and seven percent with about 3.2 percent being ideal. Vitamin E is optionally included between about 0.125 and 1.0 percent with about 0.8 being ideal.

Rose oil generally includes either rose otto (attar of rose, attar of roses) or rose absolute. It is the essential oil extracted from the petals of various types of roses. Any variety of substance generally referred to as rose oil as accepted by the US Food and Drug Administration is intended to be considered rose oil.

Olive oil generally includes the natural pressing of olives. Preferably extra virgin is utilized. However, other presses or extraction methods that may suitably produce oil from olives may be used.

Coconut oil is generally an oil extracted from the kernel or meat of coconuts harvested from the coconut palm. Beeswax is wax produced by bees or a synthetic equivalent. Beeswax may also be substituted for other waxes of similar weight and characteristics. Salicylic acid is commonly available and known. Neem oil is an extract from the neem tree. Jojoba oil is commonly available and described as an extract from the jojoba plant and seed.

The foregoing description conveys the best understanding of the objectives and advantages of the present invention. Different embodiments may be made of the inventive concept of this invention. It is to be understood that all matter disclosed herein is to be interpreted merely as illustrative, and not in a limiting sense.

What is claimed is:

1. A topical skin treatment formula for the treatment of psoriasis comprised of the following ingredients by weight blended into a homogenous mixture:
between 47-59 percent coconut oil;
between 24-37 percent cocoa butter;
between 11-19 percent olive oil;
between 0.1-1.9 percent rose oil;
between 2.9-5.6 percent bees wax;
between 0.5-2.5 percent salicylic acid;
between 0.125-1.0 percent vitamin E.

2. A topical skin treatment formula for the treatment of psoriasis as disclosed in claim 1 further characterized in that the mixture contains by weight:
50 percent coconut oil;
30 percent cocoa butter;
15 percent olive oil;
0.2 percent rose oil;
3.5 percent bees wax;
0.5 percent salicylic acid;
0.8 percent vitamin E.

3. A topical skin treatment formula for the treatment of psoriasis comprised of the following ingredients by weight blended into a homogenous mixture:
between 28-39 percent coconut oil;
between 19-28 percent cocoa butter;
between 28-39 percent olive oil;
between 0.5-1.0 percent rose oil;
between 2-7 percent bees wax;
between 0.1-1.5 percent jojoba oil
between 0.125-1.0 percent vitamin E.

4. A topical skin treatment formula for the treatment of psoriasis as disclosed in claim 3 further characterized in that the mixture contains by weight:
35 percent coconut oil;
25 percent cocoa butter;
35 percent olive oil;
0.5 percent rose oil;
2.7 percent bees wax;
1 percent jojoba oil;
0.8 percent vitamin E.

5. A topical skin treatment formula for the treatment of psoriasis comprised of the following ingredients by weight blended into a homogenous mixture:
between 19-31 percent coconut oil;
between 19-31 percent cocoa butter;
between 19-31 percent olive oil;
between 0.3-1.0 percent rose oil;
between 3.5-7 percent bees wax;
between 0.3-2 percent neem oil;
between 16-26 percent jojoba oil;
between 0.125-1.0 percent vitamin E.

6. A topical skin treatment formula for the treatment of psoriasis as disclosed in claim 5 further characterized in that the mixture contains by weight:
25 percent coconut oil;
25 percent cocoa butter;
25 percent olive oil;
0.5 percent rose oil;
3.2 percent bees wax;
20 percent neem oil;
0.5 percent jojoba oil;
0.8 percent vitamin E.
